(19) United States
(12) Patent Application Publication
(10) Pub. No.: US 2011/0077492 A1
(43) Pub. Date: Mar. 31, 2011

(54) Systems for Bidirectional Communication With A Patient Via A Medical Measurement Device

(75) Inventors: Paul M. Kapu, Nolensville, TN (US); Ray J. Davis, Denton, TX (US); Raymond W. McClure, II, Denton, TX (US)

(73) Assignee: Allegiance DMS, LLC, Houston, TX (US)

(21) Appl. No.: 12/569,275

(22) Filed: Sep. 29, 2009

(51) Int. Cl. A61B 5/145

(52) U.S. Cl. 600/365

(57) ABSTRACT

The present disclosure describes a system for providing bidirectional communication between a patient operating a medical measurement device and a third party, using the device to facilitate such communication. Some examples of the device collect data concerning the physiological condition of the patient. The device transmits the data to a DME server for subsequent transmission to the third party, and also receives treatment-related information from the DME server that is originated by the same or a different third party. The DME server transmits the data received from the device to the third party, if said third party is authorized to receive such data.

### CROSS-REFERENCE TO RELATED APPLICATIONS

The present disclosure is related to U.S. patent application Ser. No. ______ entitled “Systems for Procuring Regulatory Data from a Patient via a Medical Measurement Device,” and Ser. No. ______ entitled “Systems for Treatment-Related Product Promotion and Ordering via a Medical Measurement Device,” both filed concurrently with the present disclosure, and both of which are hereby incorporated by reference.

### BACKGROUND

Since their initial development in the early 1960s, glucose meters have evolved into relatively small but sophisticated devices. These medical measurement devices are used to determine the approximate concentration of glucose in a person's blood. The monitoring of glucose levels is an important element of managing diabetes. With the introduction in the early 1980s of home glucose meters, diabetics were provided with a tool that allowed much more frequent monitoring of glucose levels. Such frequent monitoring enables diabetics and their doctors to better adjust a patient's diet and/or medication levels in order to achieve closer-to-normal levels of glucose for as much of the time as possible. By doing so, patients can reduce both the severity and rate of occurrence of both short term and long term complications that can accompany both hyperglycemia (high blood sugar) and hypoglycemia (low blood sugar).

Many home glucose meters include programmable processors that allow them to perform a wide variety of manipulations and computations using the raw glucose level data collected. Small and inexpensive non-volatile memories (e.g., flash memories) with large storage capacities have further enabled these meters to store large numbers of samples collected over long periods of time, as well as the results of large numbers of calculations performed on the sampled data. A number of home glucose meters can be connected to a computer (e.g., a home PC), enabling the transfer of the collected and/or processed data to be stored and displayed on the computer. This enables the patient to identify trends in the variations of glucose levels over the course of the day, thus improving the patient's ability to manage and reduce such variations. The data may also be transferred to a doctor's computer, either directly or via a network (e.g., the Internet) to provide the doctor with feedback regarding the effectiveness of the treatment, as well as the level of treatment compliance on the part of the patient. Current systems, however, generally only transfer the data for manipulation, storage, statistical assessment and display by a person (patient and/or doctor). Further, the communication of the information is one way, i.e., from the meter to a computer, with no substantive information being provided back to the patient's device.

Nonetheless, home glucose monitoring has proven to be an effective tool in managing diabetes by mitigating many of the long terms complications that can be brought on by this disease. The United States government has taken note of this benefit, and both the meters themselves as well as the consumables necessary to perform the tests (e.g., glucose test strips and calibration chemicals) are covered under Medicare, such that the cost of such components are partially or completely reimbursed by the government. However, because of the high level of Medicare fraud, a significant number of verification regulations have been instituted by the government to ensure that a patient has a legitimate medical need for such a meter and its consumables.

To assist with such verification, Medicare regulations require providers of the meters and/or the consumables used by the meters (sometimes referred to as “durable medical equipment” or DME providers) to collect the required verification information, and to supply the collected information to the government, specifically the U.S. Department of Health and Human Services (HHS) that administers the Medicare program. To this end, benefit payments made under Medicare for such devices and/or consumables are made directly to the DME provider once the provider has supplied HHS with the documentation required by the Medicare regulations—for example, prescriptions for the meters and consumables, confirmation of receipt of the meter by the patient, and confirmation of completion of meter training by the patient. The DME provider is thus motivated to secure the required documentation, since the provider will not get reimbursed by HHS under Medicare without such documentation.

The DME providers have thus evolved into a central data collection point, with communication links to most, if not all, of the parties that participate in managing a patient's glucose level monitoring and corresponding treatment. Nonetheless, many of these links are not automated, and many of those that are automated are not truly bi-directional, i.e., are not links that allow for substantive information exchange to and from both communicating entities that is more than incidental communication status and control information. Existing links thus significantly limit and may even prohibit potential uses by authorized third parties of DME provider data repositories (including the DME providers themselves) and of the communication links (where they even exist) that couple the DME providers to other entities.

### BRIEF DESCRIPTION OF THE DRAWINGS

FIG. 1A illustrates an example of a system for procuring regulatory data from a patient via a medical measurement device.

FIG. 1B illustrates an example of a medical measurement device.

FIG. 2 illustrates an example of a patient data record suitable for storing regulatory data associated with the patient.

FIG. 3 illustrates an example method for confirming delivery to a patient of a medical measurement device.

FIG. 4 illustrates an example method for acknowledging and logging a message received from a medical measurement device, generating a report specific to the received message, and sending the report to HHS.

FIG. 5 illustrates an example method for initiating and processing an order for consumables associated with a medical measurement device.

FIG. 6 illustrates an example method for confirming delivery to a patient of consumables associated with a medical measurement device.

FIG. 7 illustrates an example method for confirming completion and comprehension of training provided via a medical measurement device.

FIGS. 8 and 9 illustrate example methods for implementing bidirectional communication with a medical measurement device.

FIGS. 10 and 11 illustrate example methods for providing and processing promotions and orders of treatment-related products and services associated with a medical measurement device.

### DETAILED DESCRIPTION

The present disclosure describes, amongst other details, a system for providing bidirectional communication between a patient operating a medical measurement device and a third party, using the device to facilitate such communication. The system includes a medical measurement device that collects data concerning the physiological condition of the patient. The device transmits the data to a DME server for subsequent transmission to the third party, and also receives treatment-related information from the DME server that is originated by the same or a different third party. The DME server transmits the data received from the device to the third party, if said third party is authorized to receive such data.

I. System Overview

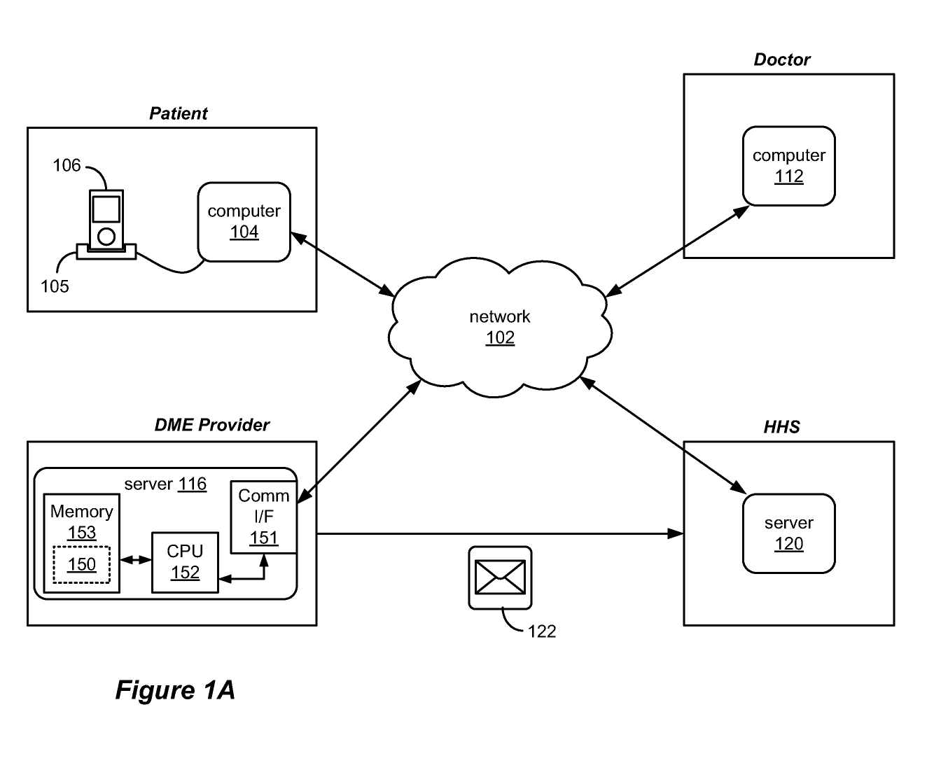

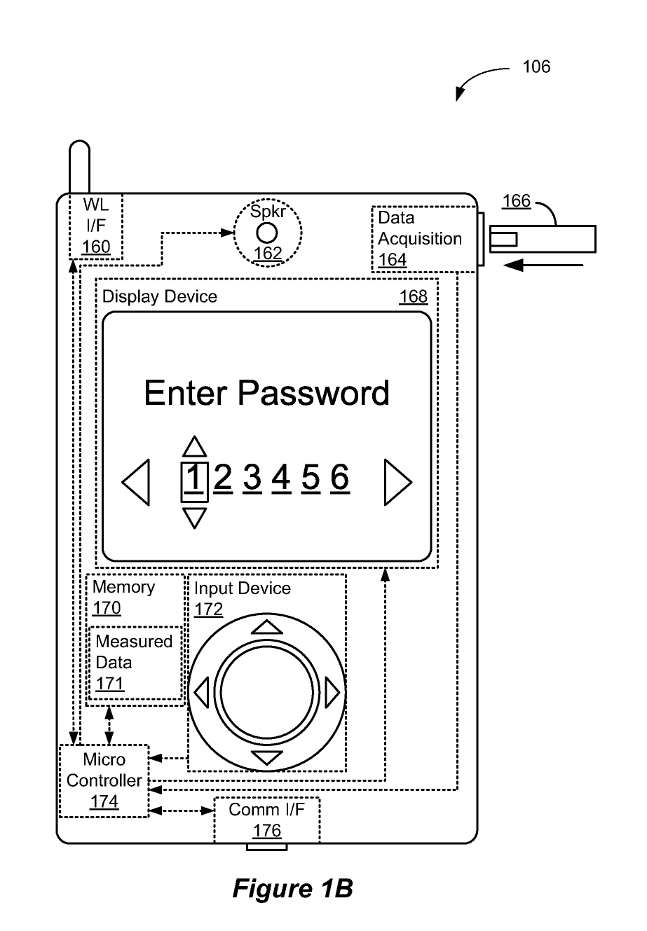

FIG. 1A shows an example of the above-mentioned system. In the example shown, a patient operates a medical measurement device 106 to measure one or more of his/her own physiological characteristics. In a specific example, medical measurement device 106 is a blood glucose level meter used by the patient to measure his or her blood glucose level. Such measurements are expected to be consistent with a doctor's prescribed treatment plan. For example, a doctor may prescribe that the patient takes his blood sugar reading at scheduled intervals, or so many times a day. As such, the glucose readings are typically time-stamped.

Such measurement data (e.g., glucose readings and time stamps) are stored in the device's memory, either in original form or as processed data to make the review of the data more clear or meaningful. The measurement data may later be retrieved from memory for additional processing, such as statistical analysis, trending and/or graphing by the patient or the patient's doctor as explained earlier. The measurement data allows a third party such as the patient's doctor, the DME provider, or HHS in its capacity as Medicare administrator, to verify compliance by the patient with the treatment plan prescribed by the doctor. The measurement data further facilitates the selection and transmission to device 106 of relevant, treatment-related promotions, as will be discussed later.

In the example shown in FIG. 1A, medical measurement device 106 communicates with a patient computer 104 via docking unit 105 by a universal serial bus (USB) connection for example. Computer 104 can be any computer device (e.g., a PC, PDA, etc.), and would typically allow for the measurement data to be additionally processed (e.g., graphed) as described above, although this is not necessary. Patient computer 104 also provides connectivity to a communication network 102 such as the Internet, and thus enables the patient to upload the measurement data stored on medical measurement device 106. Connectivity between device 106 and network 102, or between computer 104 and network 102, can also be wireless in whole or in part (e.g., Bluetooth, Wi-Fi, cellular, etc.). However, for simplicity, such wireless connectivity is not shown. As further shown in the example of FIG. 1A, the communication link between medical measurement device 106 and network 102 is bidirectional, allowing data to be transferred to or from device 106. This is in contrast to traditional medical measurement device systems that provided only a one-way communication link from the device 106 to the network 102. This bi-directional link enables information to be exchanged between the device 106 and other parties in both directions, and further allows one party to send queries to the other to request information, as is described in more detail below.

Data uploaded from the device 106 is received by a server 116 operated by the DME provider via communication interface 151 of the server. The server 116 is referred to herein as the DME server 116 for convenience, because such server may be located with or controlled by a DME provider. However, this is not strictly necessary, as the server 116 could be located elsewhere or under the control of other parties having some relation to the system 100. For example, the server 116 could be located at or controlled by an entity that supplies products or services to the DME provider, such as distributors, manufacturers, insurance companies, hospitals, HMOs, government agencies, and/or others.

The data is processed by the CPU 152 (or other similar processing logic) and stored in memory 153 within a record 150, which record 150 is explained in further detail below. Other data required by HHS for Medicare reimbursement, such as proof of delivery of the medical measurement device and of related consumables, are also stored within record 150. The data so collected and stored within the record 150 thus comprises the regulatory data used by the DME provider to support reimbursement claims submitted to HHS under Medicare for the costs of the device and its related consumables. Part or all of record 150 may be submitted for this purpose by the DME provider to HHS as a printed document via regular mail 122, or electronically via network 102, assuming HHS allows (or will eventually allow) for electronic submission of Medicare claims. The centralized location of the data on the DME provider's server facilitates convenient access to the patient's data by authorized parties. Such centralization also facilitates the implementation of security measures that limit access to only duly authorized parties, thus safeguarding the patient's privacy.

FIG. 1B illustrates an example of the hand-held medical measurement device 106 of FIG. 1A. The device 106 includes a display device 168 that can present textual and graphical information to a user, a speaker 162 for providing audio, and an input device 172 that accepts user-provided information. Thus, for example, a user may be prompted to enter a password as shown, and the user can use the left/right cursor controls of input device 172 to select which digit to enter, and the up/down cursor controls to select the value for each digit. Other examples of the device 106 may include a telephone keypad or a full QWERTY keyboard for entering alpha-numerical data and/or text, or a touch interface allowing the user to use a keypad presented on the display device 168. Microcontroller 174 generates the information displayed to the user and audio data provided through speaker 162, and also processes the information input by the user via input device 172. Microcontroller 174, which performs the data processing functions in the example of FIG. 1B, can comprise microprocessors, a field programmable gate array (FPGA) an application specific integrated circuit (ASIC), or any other logic processing device. Device 106 also includes a data acquisition module 164, which comprises the circuitry for taking the measurement data indicative of the patient's health. When device 106 comprises a glucose meter, data acquisition module 164 accepts a test strip 166 with a blood sample, determines the blood glucose level of the sample, and provides the sample glucose level as measurement data provided to microcontroller 174. The measurement data 171 is stored (or alternatively processed, then stored) by microcontroller 174 in memory 170 and may later be transmitted to DME server 116 through either communication interface 176 or wireless interface 160, both of which may generically be referred to as communication interfaces.

From this brief description of the system 100, it can be appreciated that system 100 has many beneficial uses. For example, the system may be used to collect regulatory data required under Medicare for example, and so can assist the DME provider in seeking reimbursement while minimizing or eliminating the need for “chasers” as explained earlier. The system's bi-directional communication link between the device 106 and the network 102 may also be used to provide communications between a patient and any of a number of authorized third parties, such as the patient's doctor to allow the patient and doctor to consult concerning the patient's health. Such bi-directionality may additionally be used to present at the device interactive, treatment-specific promotions to the patient. Each of these uses is described in detail below.

II. Procuring Regulatory Data

A. Pre-Approval

As already noted, the cost of medical measurement devices and related consumables may be covered, at least partially, under a governmental program such as Medicare if compliance with Medicare regulations can be shown. For example, when a DME seeks reimbursement for the cost of a blood glucose level meter, proof of a diagnosis of a medical condition that requires blood sugar level monitoring (e.g., diabetes) must be provided to HHS. Likewise, when a DME provider seeks reimbursement for consumables used by the device, such as glucose test strips, the DME provider must provide proof that that the number of consumables for which reimbursement is sought is warranted. For example, if the treatment plan for the patient requires the patient to monitor glucose levels three times per day, the DME provider will use such treatment information to prove its right to reimbursement for 90 strips per month (assuming a 30-day month). A DME provider typically provides such proof of diagnosis and the treatment plan to HHS prior to shipment of the blood glucose meter to the patient, so that the DME provider's eventual reimbursement claim for the cost of the meter, and for the costs of consumables, can be pre-approved by HHS.

Such proof may include documents from the patient's doctor describing the tests performed, the resulting diagnosis (perhaps using Healthcare Common Procedure Coding System (HCPCS) codes), the doctor's treatment plan (e.g., that the patient test his glucose three times per day), and the doctor's prescription for the medical measurement device and the consumables (e.g., glucose test strips). Such diagnosis documents can be procured by the DME provider by conventional means, such as through the use of chasers as discussed earlier. Or, system 100 can be utilized, with the patient scanning such documents on their computer 104, and sending them to the DME provider through the communication links already discussed.

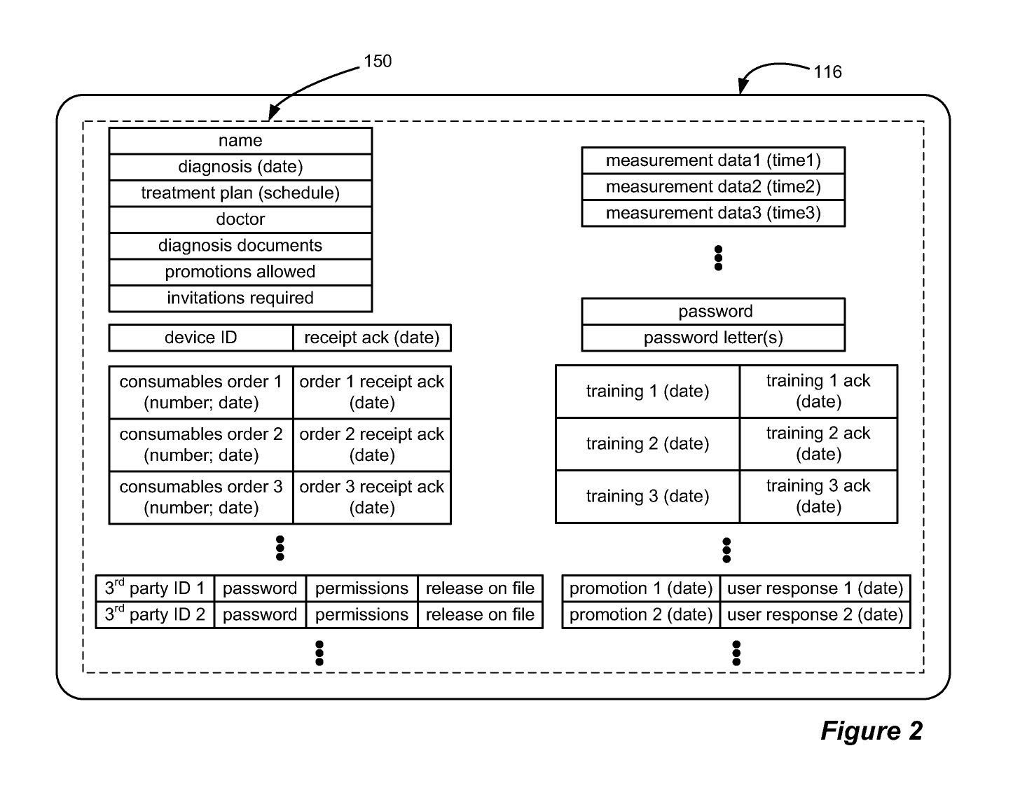

Either way, once received by the DME provider, the diagnosis documents can be used to create a record 150 in the DME server 116, as shown in FIG. 2. Although the contents of the record 150 can vary, the record 150 as illustrated initially comprises those elements necessary for pre-approval, such as the patient's name, a diagnosis (e.g., diabetes), the date of diagnosis, the prescribed treatment plan (e.g., the schedule at which the patient is to test his glucose levels, such as three times a day), and the name of the patient's doctor. The diagnosis documents in raw form (e.g., as scanned PDF files) may also be included. The record 150 for each patient will eventually be updated with additional information, some of which is shown in FIG. 2 and will be discussed further below.

Once record 150 is compiled, the DME provider can provide it to HHS for pre-approval either electronically or by physical mail as already discussed. A pre-approval report suitable for submission to HHS can also be generated from the record 150. Once pre-approval is obtained from HHS, the DME provider ships the prescribed blood glucose level meter to the patient.

B. Device Receipt

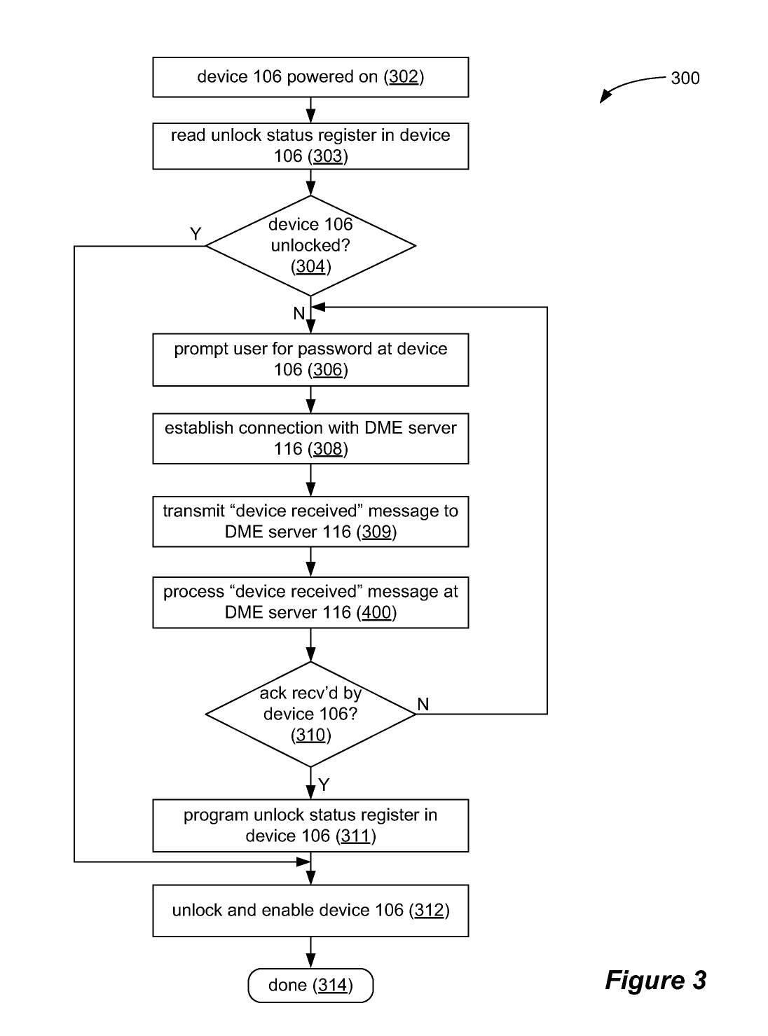

Although the DME may have the cost of the device 106 pre-approved by HHS, final payment of the DME provider's claim is not made until the DME provider provides HHS with proof of delivery of the device 106 to the patient—with such proof of delivery comprising but one kind of regulatory data required by HHS. Method 300 of FIGS. 3 illustrates how system 100 can provide such proof of delivery.

When the device 106 is shipped by the DME provider to the patient, the device 106 is “locked” such that it cannot be operated until a password is entered and transmitted back to the DME server 116. That password, along with an ID for the device shipped to the patient, is stored by the DME in the patient's record 150 (FIG. 2).

When the device 106 is powered on by the patient or the patient's representative (e.g., a nurse or relative) (302), a check is performed to determine if the device is already unlocked (304) for use. This check can comprise reading an unlock status register in the nonvolatile memory of the device 106 for example (303). If the device has already been unlocked (304), the rest of unlock method 300 is bypassed and the device is allowed to be operated normally, ending the method (314).

If the device 106 has not yet been unlocked (304), the user is prompted to enter a password using the user interface of the device 106 (306), which user interface typically comprises at least a display and input buttons. The password is preferably provided by the DME provider to the patient independently from shipment of the device, such as by a separate letter mailed to the patient. A copy of the password letter can be included by the DME provider as part of the patient's record 150 (see FIG. 2). After the password is entered, a connection between the device and the DME server 116 is established (308) if it hasn't been established already. For example, the user may be prompted to dock device 106 to patient workstation 104 which in turn connects to the DME server 116. Or, if the device 106 uses a wireless link such as those discussed earlier, the device may prompt the user to establish a network connection if such a connection is not already available and detected.

Once a network connection is established, the device 106 transmits to DME server 116 a “device received” message (309), which includes a “device received” notification, a device identifier (ID) and the entered password. The device ID is preferably written into tamper-proof memory within the device, and may be written into the device by its manufacture or by the DME provider. In either case, the ID of the device 106 provided to the patient, like the password, was previously incorporated into the patient's record 150 on the DME server 116 (FIG. 2), during the pre-approval stage or otherwise.

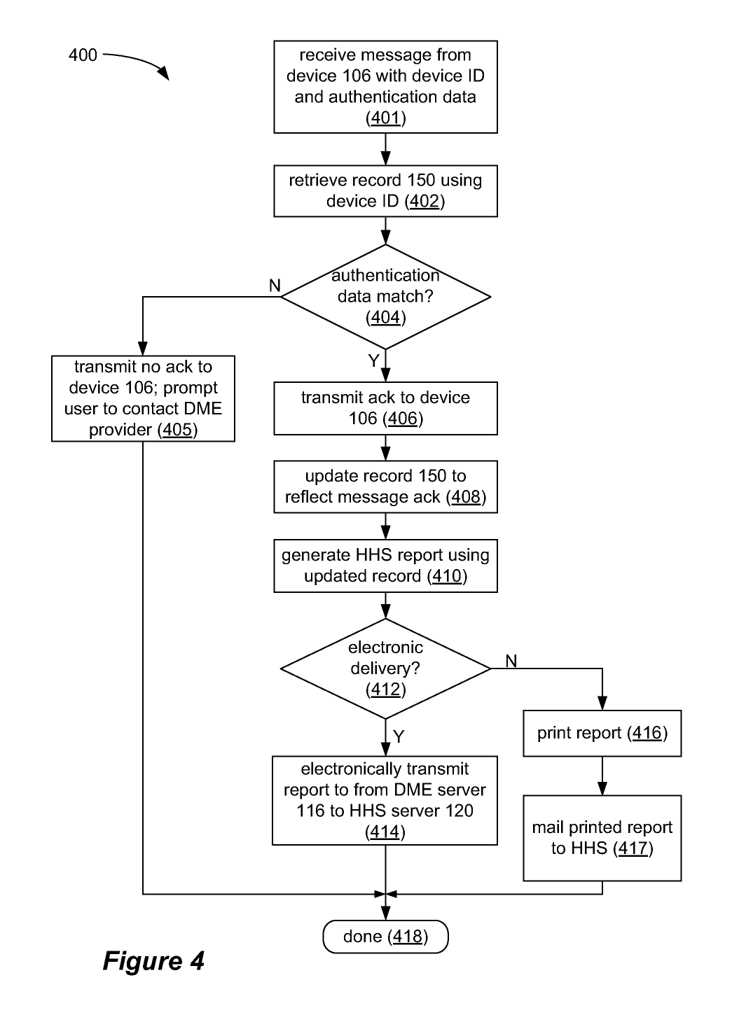

The received message is processed by the DME server 116 (400), as explained in more detail below with respect to FIG. 4. This processing includes verification by the DME server 116 of authentication data included in the message, such as the password provided by the user. Referring again to the example of FIG. 3, if the password entered by the user matches the password in the patient's record 150, the DME server 116 provides an acknowledgment (“ack”) back to the device 106. Receipt of that acknowledgment is checked at the device 106 (310). If the acknowledgment has not been received at the device 106, the device will eventually inform the patient of the same, and will prompt the patient to enter the password again (306)—i.e., method 300 repeats until a valid password corresponding to the device ID has been entered. If the acknowledgment has been received at the device 106 (310), the device programs the unlock status register to reflect an unlocked state (311). Such programming of the register will allow the device 106 to skip the unlock method when the device is powered on in the future (see steps 303 and 304). Thereafter, the device 106 is unlocked and enabled (312), and the method ends (314).

Alternatively, the password may be programmed into the device 106 by the DME provider before the device is shipped. When the user enters the password, it is verified locally by the device 106 with a verification message sent to the DME server 116 instead of the password. The DME server 116 can then issue the acknowledgment to the device 106 contingent on receipt of the verification message. Also, other types of authentication data may be used instead of, or in addition to, the password described above. Such authentication data may include, for example, biometric data such as fingerprint and retina data. The biometric data may be stored within patient record 150, and compared with biometric data collected by the device 106. For example, a microphone built into the device may be used to record the patient verbally identifying themselves, with the recording being formatted as an audio file that can be transmitted to DME server 116 (e.g., an MP3 file).

As previously noted in the example of FIG. 3, the DME server 116 receives notification of the patient's receipt of the device via a “device received” message transmitted by the device 106. FIG. 4 shows an example method 400 performed by the DME server 116 for processing such messages. After receiving a message with the device ID and authentication data (401), the DME server 116 uses the device ID to locate a record 150 containing the same device ID (402). Once the correct record 150 is located at the DME server 116, the received message is authenticated by comparing the authentication data from that record 150 (e.g., the password stored in that record) with the received authentication data (e.g., the password entered by the user). As already noted, other forms of authentication data (e.g., biometric data) may also be used. If the authentication data matches (404), an acknowledgment is sent to the device 106 (406). Furthermore, the record 150 at the DME server 116 is updated to reflect the acknowledgment and the current date (408) (e.g., “receipt ack (date)” in FIG. 2). If the authentication data doesn't match, the DME server 116 fails to transmit an acknowledgment back to the device 106 (405), e.g., by not sending any message back to the device 106, or alternatively by sending a negative acknowledgement or “nack” to the device. The DME server 116 may additionally at this point prompt the user to contact the DME provider using any reasonable means, such as by phone, mail, e-mail, or by texting using the device 106 itself.

The combined methods 300 and 400 not only provide a safeguard against unauthorized use of the medical measurement device 106, they also provide confirmation of the patient's receipt, since the DME server 116 can only generate an acknowledgment upon receipt of the correct information (e.g., device ID and password) from the device. Therefore, the updated record 150, including particularly the logged acknowledgment, can be used by the DME provider to prove receipt of the device by the patient—one piece of regulatory data needed by the DME to prove its entitlement to its Medicare reimbursement claim. The remaining steps in method 400 illustrate how the system 100 assists in generating a report, complete with proof, for a Medicare reimbursement claim relating to the cost of the device 106.

Once the record 150 has been updated to reflect transmission of the acknowledgment (408), the DME server 116 generates an HHS report (410) specific to the message received. For example, a Medicare reimbursement report is generated when a “device received” message is received and processed by the DME server 116. That report can comprise the entirety of the patient's record 150, because that record has all of the information needed to prove the DME provider's claim for Medicare reimbursement for the cost of the device, including all relevant patient data, diagnosis, and patient receipt of the device as reflected in the acknowledgment transmittal (and perhaps as bolstered by a copy of the password letter). Alternatively, the DME server 116 can process the record 150 to pick out necessary fields of data when generating the Medicare reimbursement report. This alternative can result in a report which is more summary and perhaps shorter in nature, and which may require less review of raw data by HHS when adjudging the DME provider's Medicare claim.

Regardless, once the report corresponding to the received message is generated (410), it can then be transmitted to HHS at an appropriate time when the DME is seeking reimbursement. In this regard, it is useful to know whether HHS can receive the report electronically (412). If so, the report is electronically transmitted from DME server 116 to HHS server 120 via network 102 (414). If not, the report is printed (416) so it can be physically mailed to HHS (417). Alternatively, the report can be saved in electronic form to a physical media, such as a CD-ROM, which may also be physically mailed to HHS if/when reports are accepted by HHS in such a format.

C. Consumables

As already noted, the cost of consumables used with the medical measurement device 106, such as glucose test strips, is also covered by Medicare, and reimbursement may be pre-approved. However, the DME provider ultimately has to prove to HHS that such consumables are warranted, and have been delivered to the patient. System 100 allows for the provision of such regulatory data relating to consumables. Additionally, system 100 provides convenience to the user in ordering such consumables by automating the ordering process. The two-way communication link between the device 106 and the network 102 facilitates such functionality.

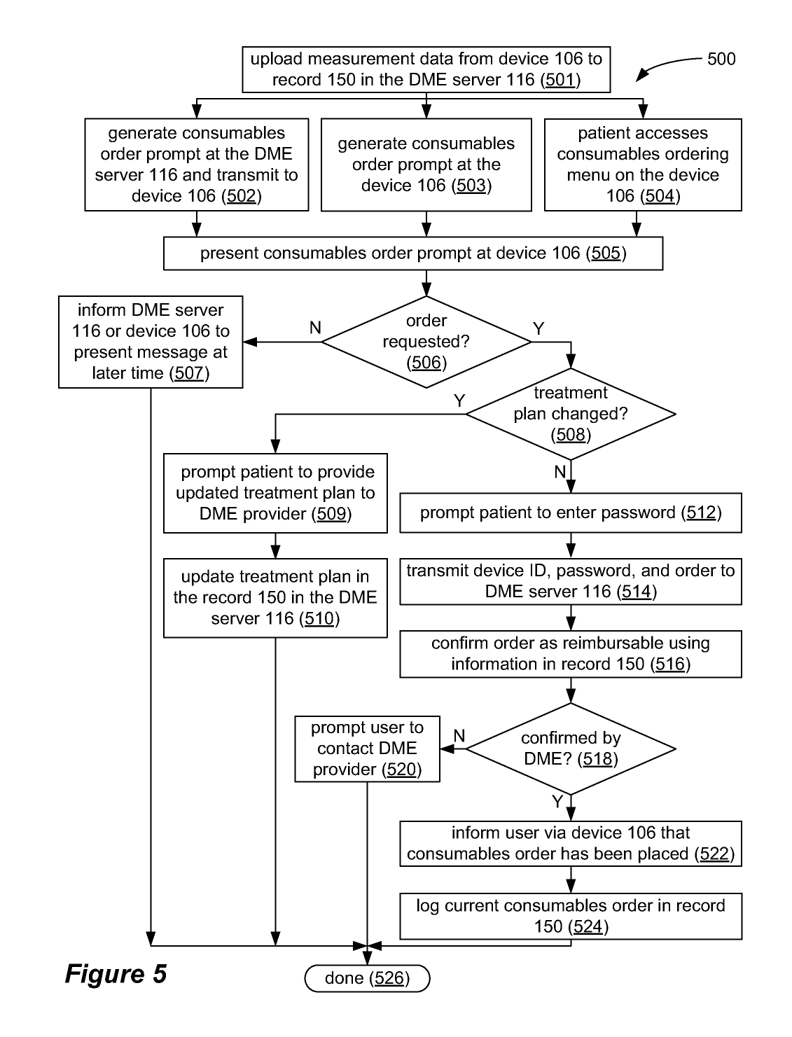

An example consumables ordering method 500 using system 100 is illustrated in FIG. 5. As a first step, a connection is established between the device 106 and the DME server 116 in any of the ways previously mentioned (by docking, wireless connection, etc.), and the measurement data in the device 106 is uploaded to the DME server 116 and stored in the patient's record 150 (501). As discussed earlier, such measurement data can include (at least) glucose readings and the time at which such readings were taken (see FIG. 2, “measurement data x (time x)”). Although this step could occur later in the process, performing it initially is convenient. As will be seen below, having up-to-date measurement data reflecting the patient's usage of the device 106 assists in automating the consumables ordering process, and in proving the DME provider's right to seeking Medicare reimbursement for the consumables. (Uploading of the measurement data is useful in other aspects of the system as well, as will be discussed in further detail later).

After the patient's record 150 is updated, the process proceeds to the generation of a consumables order prompt, which can be initiated in several ways illustrated in steps 502-504 of FIG. 5. In step 502 the DME server 116 generates a consumables order prompt. Such prompt is preferably generated when a patient is likely in need of ordering additional consumables. For example, the patient record 150 (FIG. 2) in the DME server 116 contains information concerning when consumables were last ordered, and how many were ordered at that time (see “consumables order x (number; date)” in FIG. 2). Record 150 also contains the treatment plan which specifies how often the patient is to use the device 106 to take a measurement, as well as the actual measurement data uploaded by the patient. From this information, the DME server 116 can anticipate when a patient should be ready to order more consumables, and can automatically generate a consumables order prompt. Once a connection between the device 106 and the DME server 116 is established, that consumables order prompt can be transmitted from the DME server 116 to the device 106 (502).

The device 106 can also anticipate when a patient should be ready to order more consumables, and can locally generate a consumables order prompt, as set forth in step 503. In this regard, the DME server's record 150, or portions of it, can be transferred to the device 106 to in effect store a shadow copy of the record on the device. Such transfer of the record 150 can occur periodically, such as every time a connection between the device 106 and the DME server 116 is established. The device 106 can then generate the order prompt in the same manner just discussed with respect to the DME server 116 (505). Additionally, the device 106 can keep count of the number of times a measurement has been taken by the patient, and can be programmed with the amount of consumables that have been ordered by the patient. In any event, the device 106 in step 503 automatically generates a consumables order prompt at an appropriate time.

The patient can also attempt to order consumables at any time and without the benefit of the generation of an automatic consumables order prompt, as set forth in step 504. In step 504, the patient simply accesses a consumables ordering menu on the device 106, so that the patient can input his consumables order manually. Regardless of the ways in which ordering in instigated, the patient is presented with a consumables order prompt in step 505. Such message may be presented on the display of the device 106, and invites the patient to order more consumables using his device 106. For example, the device 106 may display the message “would you like to order 50 more glucose test strips now,” and provide user selections for “yes” or “no.” (Fifty strips are used in this example because they are normally packaged in this amount. However, another number could be specified).

If the user opts to postpone the order by choosing “no” (506), the system 100 (i.e., either the DME server 116 or the device 106 depending on which automatically generated the message) will store such fact and re-present the message to the patient at some later time (for example, a few days) (507), ending the consumables order processing (526) without placing an order. However, if the patent decides to order the consumables (506), the system 100 (i.e., DME server 116 or device 106) next inquires whether the patient's treatment plan has changed (508). This could occur for example if the patient's doctor has now prescribed the patient to take his glucose readings more frequently. If the user does indicate a change in the treatment plan, the device 106 prompts the patient to provide an updated treatment plan prescription to the DME provider (509). This can occur in several different ways: for example, the patient can mail his treatment plan prescription to the DME provider, or can use his device 106 or computer 104 to provide a scanned copy of his new prescription to the DME server 116.

In any event, once the DME provider has received this new treatment plan prescription, the DME provider can update the patient's record 150 in the DME server 116 (510). In the example shown, this update to the patient's record completes the consumables order processing (526) without placing the order or re-prompting the user to place an order. This may be done to allow the DME provider to obtain independent verification of the prescription change. Once such verification is obtained, a new consumables order prompt may be generated (e.g., by the DME server 116) the next time measurement data is uploaded (i.e., the next time method 500 is performed).

If the user indicates that the prescription has not changed (508), the method 500 prompts the user to enter his password (512), which can be the same password used to unlock the device as previously discussed. As before, other types of authentication data may also or alternatively be used if supported by the system 100. The device ID (coded into the device as discussed earlier), the password, and the order are then transmitted from the device 106 to the DME server 116 (514), with the password being used as before to authenticate the transaction.

At this point, method 500 optionally allows the DME server 116 to confirm that the received order is reasonable and therefore will likely be reimbursed by HHS at step 516. This step 516 is particular useful in the event that the user decides of his own accord to order consumables using device 106 (step 504). By contrast, if the DME server 116 or the device 106 has automatically generated the consumables order request (steps 502, 503) using the data in the patient's record 150, it can be assumed that the number and timing of the consumables order is appropriate and hence likely reimbursable through Medicare, because data relevant to proving reimbursement (i.e., last order specifics, treatment, plan, actual consumables usage, etc.) has been acquired and scrutinized in earlier steps.

Therefore, optional step 516 automatically confirms the order by comparing the order to the data in record 150. For example, assume the patient tries to order 50 glucose test strips, but his record 150 confirms that 50 glucose test strips were delivered to him last week and that he is only prescribed to test three times a day. In such a case, automatic comparison at the DME server 116 would reflect that the patient is not in need of further glucose test strips at this time, and therefore that if the DME provider fills this order that HHS would likely not reimburse the DME provider. Accordingly, the confirmation at step 518 would fail, and the patient would be prompted to contact the DME provider (520) through any reasonable means such as those already discussed. The consumables order processing is thus again ended (526) without placing the order.

Should the order be confirmed at step 518, the user is informed using the display on his device 106 that the order has been placed (522). This current consumables order, including the number of consumables ordered, is then logged in the patient's record 150 (524) as the next sequential consumables order (see “consumables order X (number)” in FIG. 2), completing the processing or the order (526). The “date” parameters in this record entry will be entered when the order actually ships, as will be discussed below.

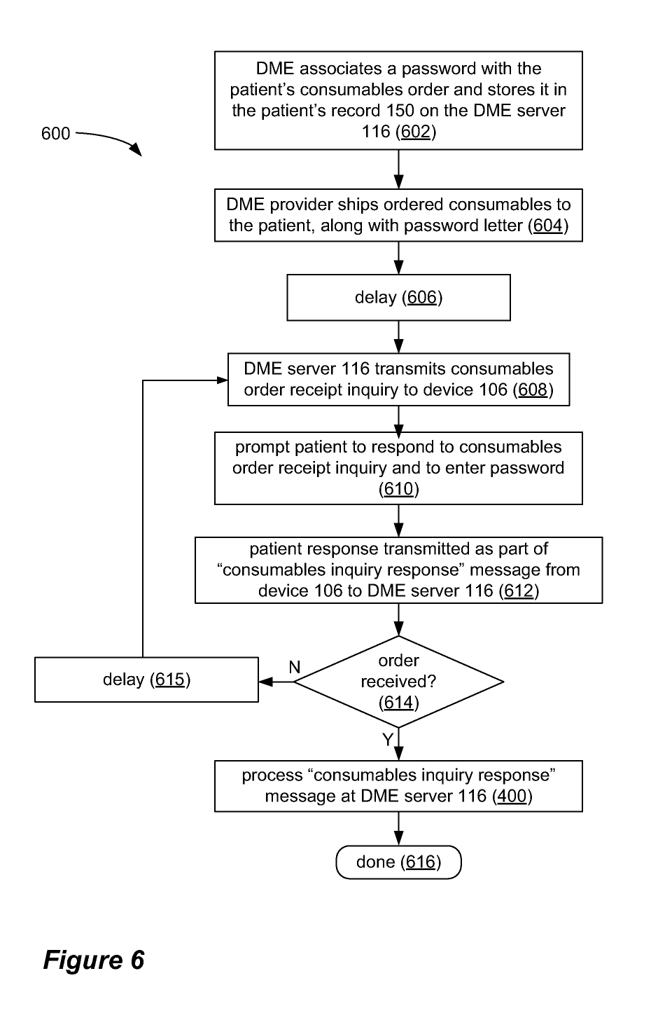

As previously noted, once an order for consumables has been placed, the DME provider must secure proof of delivery of the consumables to the patient in order to secure reimbursement from HHS under Medicare. FIG. 6 illustrates a method 600 by which system 100 can procure the required proof of delivery of consumables, such as those ordered by method 500. Steps 602 and 604 represent preliminary steps in which the ordered consumables are actually sent to the patient. First, the DME provider associates a password with the patient's pending consumables order and stores that password in the patient's record 150 in the DME server 116 (602). Such password can overwrite previous passwords in the record (e.g., the device unlock password discussed earlier), or can be added thereto and specifically associated with the present consumables order. Although not shown, at this point, the DME provider could seek pre-approval from HHS for reimbursement of the to-be-shipped order. However, because earlier steps in the process have already addressed procurement and review of necessary proof for a consumables reimbursement claim, seeking pre-approval at this stage would not be necessary, and thus is not illustrated. Next, the consumables are mailed to the patient by the DME provider, along with a letter informing the patient of the password associated with the consumables order (604). At this point, the DME provider would update the record 150 to reflect the date of shipment. The letter and consumables could also be mailed separately for additional security.

After some delay (606) to allow for shipping (perhaps a few days), and upon sensing a connection with the device 106 if not already established, the DME server 116 transmits a consumables order receipt inquiry to the patient's device 106 (608), which inquiry is then presented to the patient (610) using the device's display for example. For example, the device 106 may ask the patient “have you received glucose test strips shipped on [date], yes or no?” The patient responds to this inquiry via the user interface of his device 106, and is subsequently prompted to enter the password associated with the delivered consumables. After the password is entered, the response and password are transmitted back to the DME server 116 as part of a “consumables inquiry response” message (612). If the patient cannot confirm receipt, another delay is instituted (perhaps a day or so), and then the method 600 essentially repeats by eventually re-presenting another consumables order receipt inquiry to the device 106 (608). If however the patient does confirm receipt, the “consumables inquiry response” message is further processed by the DME server 116 according to method 400 of FIG. 4, thus successfully completing the procurement of the required proof of delivery of the consumables (616). The message processing performed by method 400 is similar to that previously described with respect to the “device received” message of FIG. 3, except that the report generated and sent to HHS is a Medicare reimbursement report supporting a claim for the cost of the consumables, rather than a report supporting a claim for the cost of the device.

D. Training

It can also be important to the DME provider when seeking Medicare reimbursement to prove to HHS that the patient knows how to use the medical measurement device, which can require proof that the patient or his representative has been trained to use the device. Such proof of training, like the device delivery and consumables delivery proof discussed previously, can be included in a patient's record 150 at the DME server 116, and can be included in a Medicare reimbursement report, such as those discussed earlier. Even if the DME is not seeking reimbursement at a given time, it may still wish to send a report confirming training to HHS as a compliance measure or to assist in the processing of reimbursement reports to be sent in the future.

The bi-directional nature of the communication link to the device 106 makes it possible for the DME provider to send training to the patient's device 106, which training can be textual, audial, or audio-visual. Video training is illustrated below as one example. Such training may be reviewed by the patient only once (e.g., upon unlocking the device 106 for the first time) or periodically (e.g., as the DME provider provides additional functionality to the device through software upgrades).

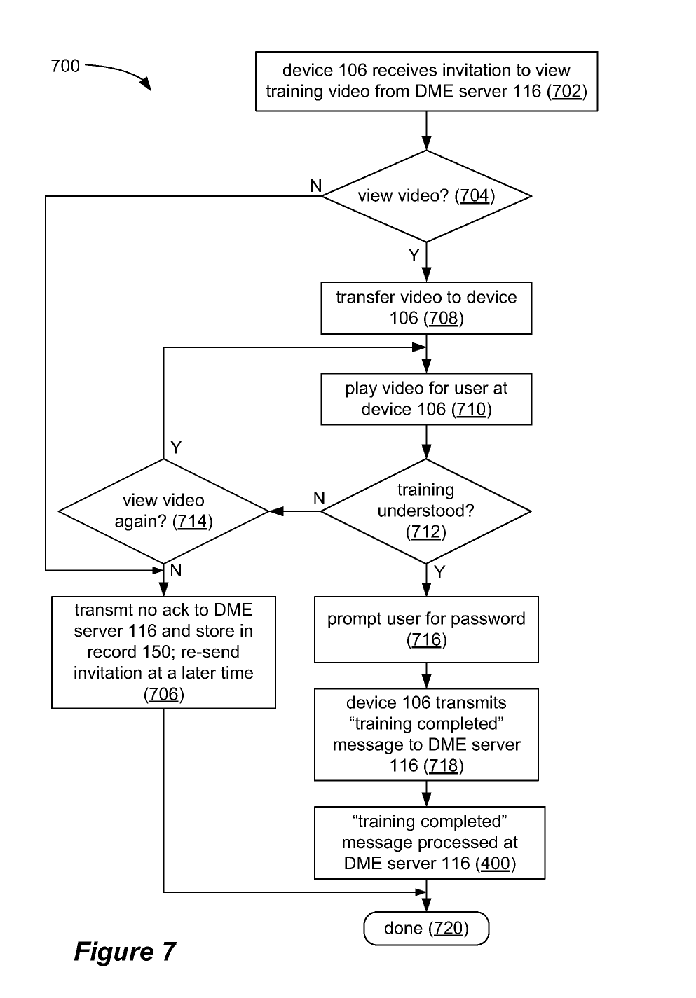

FIG. 7 illustrates a method 700 that prompts a user to view a training video and stores confirmation at the DME server 116 that the video was in fact reviewed and understood. In step 702, the device 106 receives an invitation to view a training video from the DME server 116. Such invitation could also come from the logic in the device 106 itself, and could come at any logical time useful to serve the user's desire for training or the DME provider's desire for proof of confirmation of training as necessary for proving a right to reimbursement. The timing of such invitation could also relate to when the user last completed training as reflected in the record 150 (see “training x (date)” in FIG. 2).

At step 704, the device 106 prompts the user to view the training video on his device 106. If the user refuses, a no-acknowledgment is stored in the patient's record 150 (e.g., “training X ack” is not flagged in FIG. 2), and the DME server 116 will re-send the invitation at a later time (706), ending the method (720). If the user accepts the invitation, the DME server 116 transfers the training video to the device 106 (708). Alternatively, the training video could be recalled from the device 106′s memory if it had been previously stored there. Transferring the video can occur by downloading a video file (such as a *.WMV or *.MP4 file) to the device 106 for playback or by streaming the video to the device 106. In either case, the transferred video is played on the user's device 106 (710) using the device's display for example.

After completion, the user is prompted by the DME server 116 to confirm whether the training was understood (712). If the user indicates using his device 106 that the training was not understood, the user is asked whether he wishes to view the training video again (714). If the user indicates “no,” then a no-acknowledgment is stored in the patient's record 150 as described earlier, ending the method (720). If the user indicates “yes,” the video is once again played. After indicating at the device 106 that he understood the training (712), the user is prompted for a password (716), and once entered a “training completed” message is transmitted from the device 106 to the DME server 116 (718). The message is further processed by the DME server 116 according to method 400 of FIG. 4, thus successfully completing the procurement of the required proof of training (720). The “training completed” message is processed by method 400 in a manner similar to that previously described with respect to the messages of FIGS. 3 and 6, except that the report generated and sent to HHS is a Medicare training compliance report, rather than a report supporting a specific reimbursement claim. The acknowledgement of the training completion is stored in the patient's record 150 by method 400 (see step 408 of FIG. 4). For example, and referring to FIG. 2, “training X ack” is now flagged, and the date of the acknowledgment of training completion is also stored. Because training can occur more than once, the patient's record 150 as shown in FIG. 2 can comprise several entries regarding different training videos (“training X”), each having their own separate acknowledgments (“training X ack”).

III. Bidirectional Communication

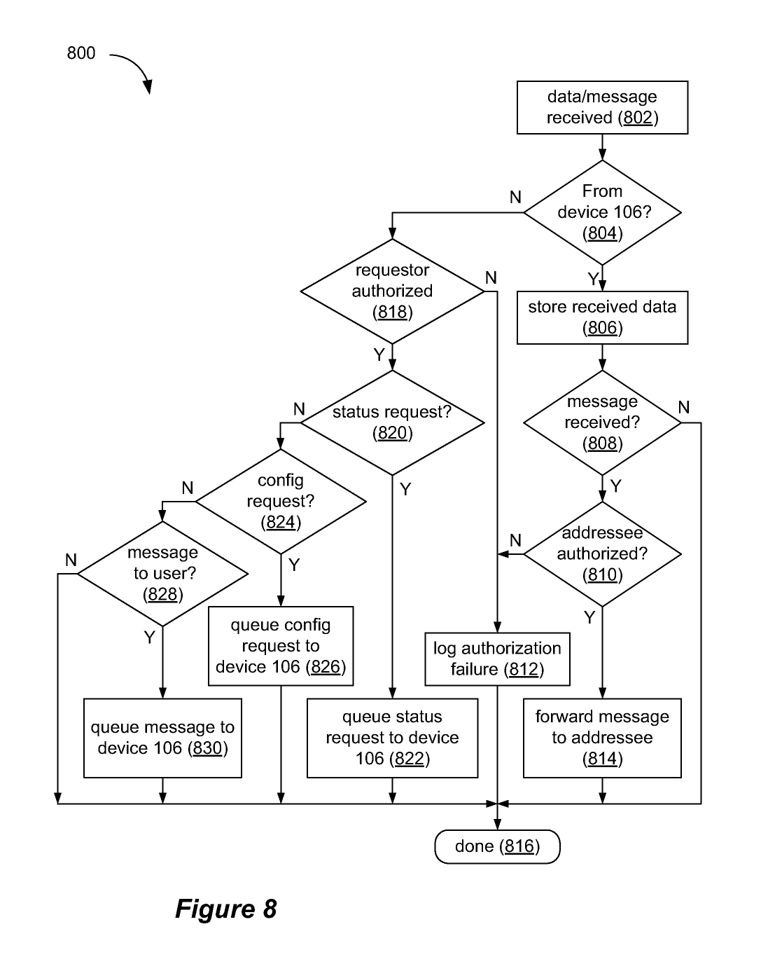

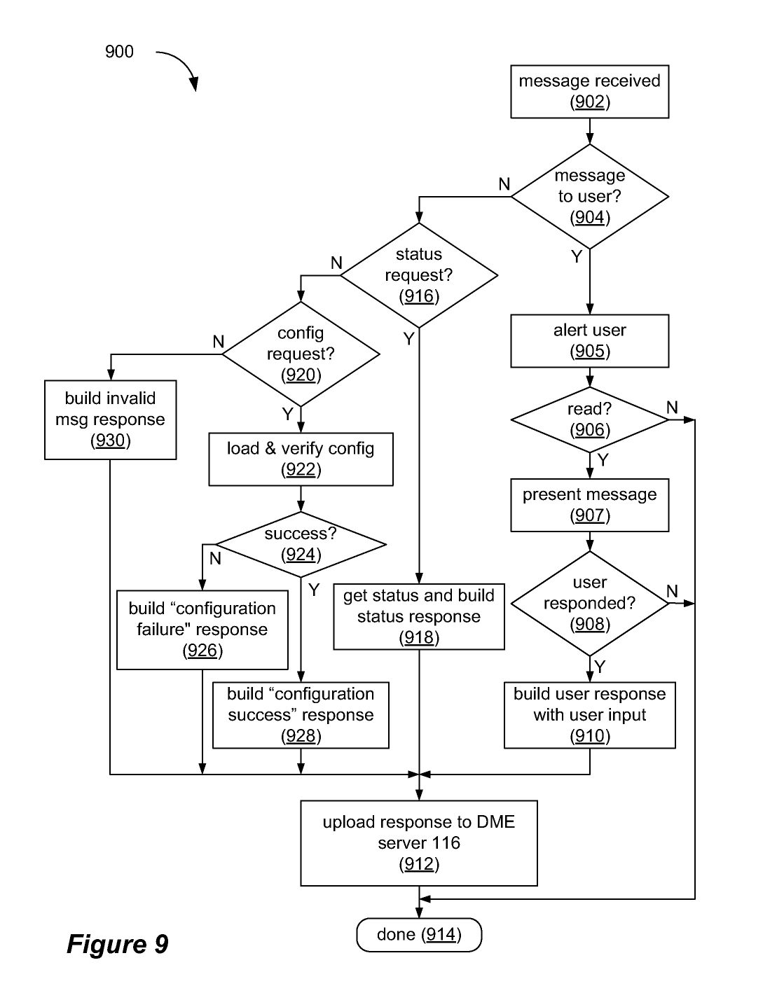

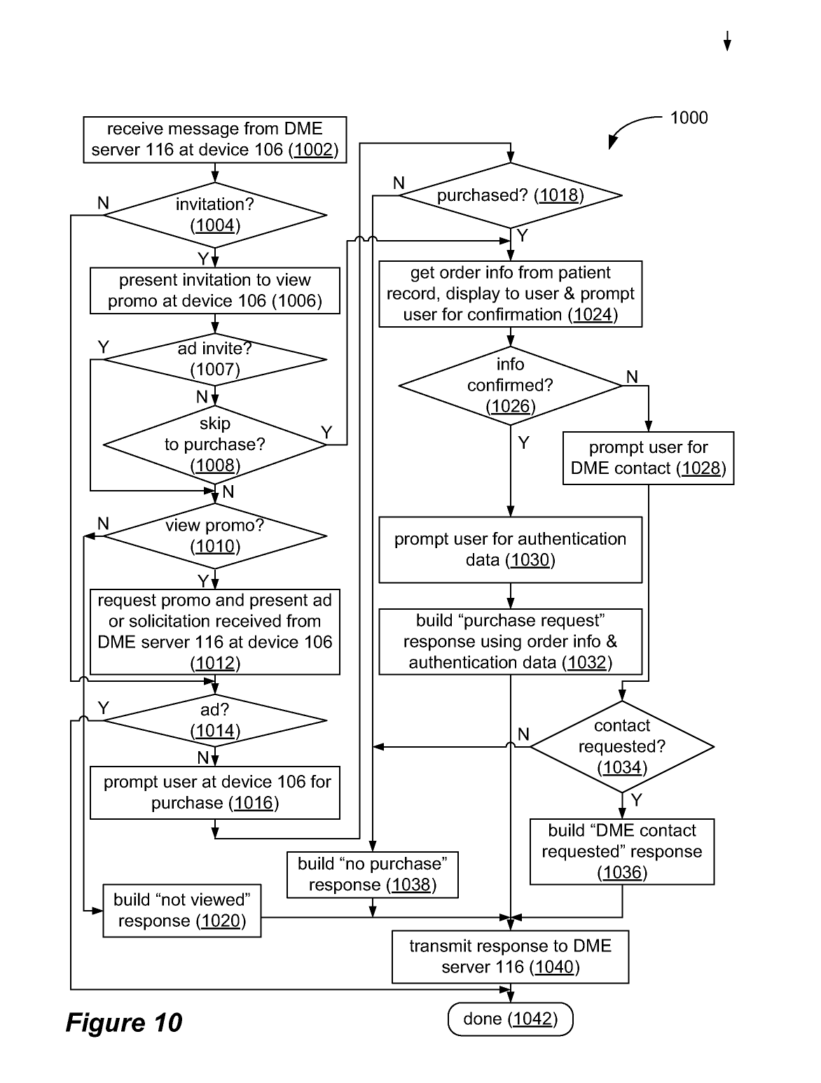

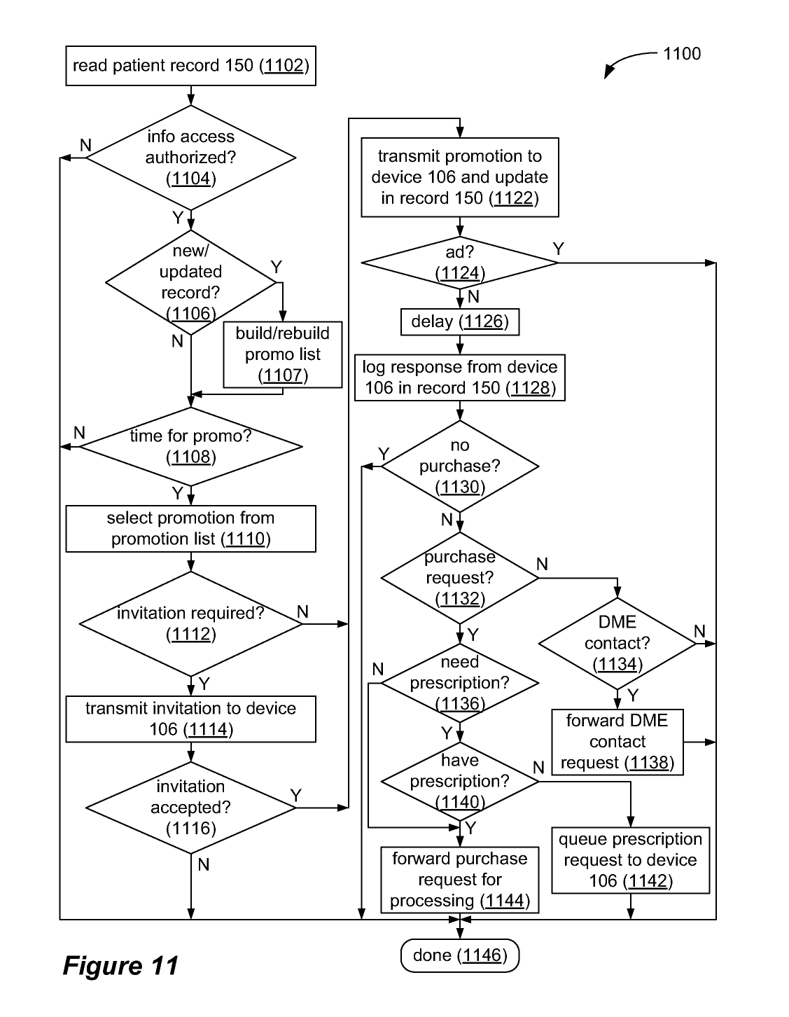

Because the communication links in system 100 are bidirectional, information can be provided between the devices, computers and servers shown, and thus between the patient operating a device and various third parties operating a computer and/or server. Some examples of the information that may be exchanged include messages to and from a patient, doctor alerts and reminders, device status data, and device configuration data. Flow charts depicting the flow of such information between third parties and the patient in the system 100 are found in FIGS. 8 and 9, with FIG. 8 depicting server-side processing, and FIG. 9 depicting device-side processing. The bidirectional communication links also enables the DME provider to present interactive, treatment-related promotions to a user of device 106. Flow charts depicting the flow of these promotions, and the processing of the users responses to them, are found in FIGS. 10 and 11, with FIG. 10 depicting device-side processing, and FIG. 11 depicting server-side processing.

A. Third Party/Patient Communication

1. Doctor/Patient Message Exchange

The bidirectional communication link between device 106 and the other elements of the system 100 of Figure lA makes it possible for a patient operating device 106 and a doctor operating a computer 112 to request information from each other, and to provide responses to such requests. Thus, for example, a doctor using computer 112 to review a patient's uploaded measurement data may notice that every morning at breakfast the patient's glucose level is spiking quickly upward. The doctor generates an inquiry at computer 112 directed to the patient, asking for more details about what the patient is eating for breakfast. Upon sending the inquiry, the doctor is prompted to enter authentication data, such as a user ID and password at computer 112. The authentication data is later used by the DME server 116 to verify that the doctor is authorized to communicate with the patient, as described in more detail below. Once the authentication data has been entered, that authentication data and the doctor's inquiry is uploaded to DME server 116.

Referring to the example method 800 of FIG. 8, the uploaded message from the patient's doctor is received by DME server 116 (802) and the message is identified as originating from a source other than device 106 (804). Since a message source other than the device cannot necessarily be treated as a trusted source, the authentication data included with the message is used to verify that the requestor is authorized to communicate with the patient (818). For example, the user ID included with the message may be checked against a list of third party IDs included within the patient's record 150 (see FIG. 2), and the password then checked to confirm that the correct password has been provided for that user ID. If it is determined that the requestor is not authorized (e.g., the user ID is not listed or the password provided is incorrect), the authorization failure is logged by the DME server 116 (812), ending the processing of the received message (816). As with patient authentication data, authentication data other than, or in addition to, a password may be used to determine if the requestor is authorized.

If the requestor is authorized to communicate with the patient, the message is next checked to determine whether it is a status request message (820) or a configuration request message (824). These types of messages are each described further below. In the present example, however, the received message is a message directed to the user (828) that is queued for subsequent transmission to device 106 (830), ending the processing of the message (816). If the type of the received message cannot be identified (i.e., not a status request, configuration request or a message to a user), the message is ignored (828), also ending the processing of the message (816).

When the medical measurement device 106 is connected to DME server 116, any previously queued messages and/or requests are transmitted to the device 106 (not shown). Referring now to the method 900 of FIG. 9, and continuing to use the example of an inquiry sent by the patient's doctor, the device 106 receives from the DME server 116 the message with the doctor's inquiry (902), which is identified as a message to a user of the device (904), e.g., the patient. Receipt of the user message causes an alert to issue at the device 106 (905). The alert may include a prompt asking the user whether he wishes to read the message now or later. If the user chooses not to read the message at that time (906), processing of the received message ends (914).

If the user of the device chooses to read the message with the inquiry from the doctor, the user will be prompted to respond to the message (907). The user may choose not to respond at that time (908), ending the processing of the received message (914). If the user does choose to respond to the message (908), a user response message is built from input provided by the user (910). The response is then uploaded from the device to DME server 116 (912), completing the method 900. The response is later transferred to doctor computer 112 (not shown). The doctor can then respond accordingly after assessing the patient's response, using the mechanisms previously described to send a message. Moreover, if part of the same communication session, the doctor may not need to enter a password again.

Similarly, a patient may send a message to the doctor to ask questions or request additional information. When connected to the DME server 116, any message originated at the device 106 (not shown) is also uploaded. Measurement data may also be uploaded at this time. Referring again to FIG. 8, upon receipt of the upload (802), the source of the data is checked (804), and if the device 106 is the source, any received measurement data is stored (806) in patient record 150 (see FIG. 2). If no message from the patient has been received together with the uploaded data (808), processing of the received data ends (816).

If a message has been received (808), authentication data within the received message similar is checked to determine if the addressee of the message is authorized to communicate with the patient (810). If the addressee is not authorized, the authorization failure is logged by the DME server 116 (812), ending the processing of the uploaded data and message (816). If the addressee is authorized (810), the message is forwarded by the DME server 116 to the addressee (814), also ending the processing of the message (816). When the message is later received at computer 112 (not shown), the doctor will be notified of the received message and prompted for a response.

Messages may comprise or be accompanied by alerts and/or reminders from the doctor to the patient. Thus, if in the previous example a doctor notices that a patient's blood glucose level has been too low for too long, the doctor can upload a message to the DME server 116 to alert the patient of the problem, and to have the patient contact the doctor as soon as possible. The message is uploaded and processed by DME server 116 using the mechanisms already described above and shown in FIG. 8. Assuming the device 106 is coupled to the DME server 116, the message from the doctor will be received and processed by the device 106 (per FIG. 9) and may additionally cause an alert to be displayed on device 106. Such an alert may be a message shown on the device display with one or more flashing indicators, an audible alarm, a spoken message played through the device's speaker, etc. For less urgent matters such as appointment reminders, less drastic indicators may be used.

2. Doctor Device Management

In addition to enabling the exchange of patient/doctor messages, the bidirectional communication links of device 106 also enable the device's status to be reported, and remote configuring of the device. For example, a doctor may wish to verify that the device 106 is in working order, and that it is configured properly and in a manner that is consistent with the prescribed treatment. To do so, the doctor causes computer 112 to send a status request message for device 106 to DME server 116.

Referring once again to FIG. 8, upon receipt of the status request message (802), DME server 116 determines that the request is not from the device 106 (804), and checks to see if the requestor (e.g., the doctor issuing the request from computer 112) is authorized to access data on the patient's device (818). The authorization check is performed in the same manner as described above with respect to patient/doctor messages. If the requestor is authorized (818), and the message is a status request message (820), a status request is queued for transmission to the device 106 (822), ending the process (816) at the DME server 116.

When the device 106 is connected to DME server 116, the previously queued status request message initiated by the doctor is transmitted from the DME server 116 to the device 106 (not shown). Referring again to FIG. 9, when received by the device 106 (902), the message is identified not as a message to the device user (904), but instead as a status request message (916). The device 106 collects the requested status data, which includes device state information (i.e., parameters indicative of how the device 106 is currently operating), as well as device configuration information (i.e., parameters indicative of how the device 106 has been set up to operate). The collected status data is used to build a response to the status request message (918), which is uploaded to the DME server 116 for subsequent transmission to the requestor (912), ending the processing of the status request by the device 106 (914). In other examples of the system 100, the status response may be uploaded by the device 106 directly to the requestor (e.g., to the doctor's computer 112).

The patient's doctor may also use computer 112 to send configuration requests for device 106 to DME server 116. Such requests allow the doctor to remotely setup or modify the configuration of the device 106 so that the device operates properly and in a manner consistent with the treatment of the patient. Referring again to FIG. 8, when the DME server 116 receives a configuration request message, the message is processed in a manner similar to that already described with regard to a status request message. Once the received message is identified as a configuration request message (824), a configuration request is queued for transmission to device 106 (826), ending the processing of the configuration request message by the DME server 116 (816).

When device 106 is connected to DME server 116, the previously queued configuration request message initiated by the doctor is transmitted from the DME server 116 to the device 106 (not shown). Referring again to FIG. 9, the initial processing of the configuration request message is similar to that of the previously described status request message. Once the received message is identified as a configuration request message (920), the configuration is loaded onto the device 106 and verified (922). Verification may be performed, for example by reading back the loaded configuration parameters and verifying that the values of the parameters are correct. If the new configuration is not successfully loaded (924), a “configuration failure” response is built (926). Similarly, if the new configuration is successfully loaded, a “configuration success” response is built (928). In either case, once a response is built, the configuration response (indicating failure or success) is uploaded to the DME server 116 (912) for subsequent transmission to computer 112, ending the processing of the configuration request by the device 106 (914). As with the status response, the configuration response may also alternatively be transmitted from device 106 directly to computer 112. If the received message cannot is not identified as a configuration request (920), and is thus a not recognized message, an “invalid message” response is built (930) and uploaded (912), ending the processing of the received message (914).

In summary, the bidirectional communication capabilities of the system 100 can be used to ensure that the device 106 is operating properly, and to make corrections to the device 106 if necessary. Thus, for example, the doctor can verify that alarm threshold levels of a patient's home glucose meter are set to the proper levels for that particular patient. Similarly, other historical parameters, such as when the medical measurement device was last calibrated, may be reviewed to ensure that the data provided by the device 106 is reliable. It should be recognized that a wide variety of device status, state, configuration and calibration parameters may be accessed in the manner described above, and access to all such parameters are contemplated by the present disclosure.

3. Communication with Other Third Parties

Although the exchanges of data described thus far have been between a doctor and a patient/user, authorized third parties other than a patient's doctor may also use the system 100 to exchange data with the patient/user and access the device in the manner described. Such authorized third parties may include, for example, a family member or a private in-home caregiver, or organizations such as the DME provider, private insurance companies, or employers. It should be recognized that any number and any type of authorized individuals and/or organizations may access some or all of a patient's information using the systems and methods disclosed herein.

B. Treatment-Related Promotions

As previously noted, a significant amount of useful data is collected and stored in the patient records 150 at the DME server 116. Such data has uses beyond regulatory reimbursement and monitoring a patient's health. For example, promotions such as advertisements for products and services may be presented by the DME provider to a user of the device 106, based at least in part on the information contained in the patient's record 150. Because of the bidirectional nature of the communication link between the device 106 and the DME server 116, it is also possible to present interactive promotions at the device 106 in the form of a product or service solicitation. Unlike an advertisement, which merely describes products and services to the user, a solicitation offers the user the option of using the device 106 to purchase the product or service described. The bidirectional communication link between the device and the DME server also facilitates the use of interactive invitations that present the user with the option of whether to view an advertisement or a solicitation. FIG. 10 illustrates an example of how the device 106 processes such invitations, advertisements and solicitations, while FIG. 11 illustrates an example of how the DME server 116 generates the invitations, advertisements and solicitations and processes the responses to the invitations and solicitations received from the device 106.

Referring now to the example method 1000 of FIG. 10, device 106 receives a message from the DME server 116 (1002). If the message is an invitation to view a promotion (1004), the invitation is presented to the user at the device 106 (1006). As will be discussed in further detail with respect to server processing in FIG. 11, the promotion can be relevant to a patient's medical condition, as reflected in record 150. For example, upon noting that the patient is a diabetic who uses a glucose meter 106, the promotion may advertise and optionally offer to sell lancets used by the patient to prick their fingers to provide the needed blood samples. Or, the promotion may advertise and offer diabetic socks or shoes. Thus, the promotions may involve products or services over and beyond those required by the patient's prescription.

The invitation presented may include a brief description of the product or service advertised or offered. If the invitation is for a solicitation and not an advertisement (1007), the user is also given the option to skip directly to the purchase of the product or service offered (1008). Invitations to view both advertisements and solicitations provide the user with the option to view the promotion or decline the invitation altogether (1010). If the user chooses to decline the invitation, a “not viewed” response is built (1020) and transmitted to the DME server 116 (1040), ending the method (1042). By tracking which solicitations are rejected, the DME provider can further refine the selection of promotions presented to the user and thus more precisely target the needs of the patient, as described in more detail below.

If the user of the device 106 accepts the invitation (1010), a request for the promotion is sent by the device 106 to the DME server 116. An advertisement or solicitation is received by the device 106 from the DME server 116 and presented to the user (1012), or alternatively streamed to the device. The received advertisement or solicitation may comprise a video file (e.g., a *MWV or *.MP4 file), which is presented to the user as video on the device screen, audio through the device speaker, or both. Alternatively, the video file may have been pre-stored locally at the device 106, in which case it is retrieved and played locally. If an advertisement is presented at the device 106 (1014), no further processing is required once the presentation is complete, and the method ends (1042). If a solicitation is presented (1014), after the presentation completes the user is offered the option to use the device 106 to purchase the advertised product or service (1016). The offer may include pricing that reflects the actual cost to the patient, based at least in part upon the patient's insurance coverage such as that provided by Medicare, as described in more detail below. If the user declines the offer (1018), a “no purchase” response is built (1038) and sent from the device 106 to the DME server 116 (1040), completing the method (1042).

If the user accepts the offer (1018), or has skipped directly to the purchase of the product or service offered (1008), information needed to place the order is retrieved from patient record 150 (1024). The record 150 can either comprise the master record stored in the DME server 116, or the local “shadow” copy stored at the device 106. If present, the record 150 can also be used to determine a suitable number of products to offer to the patient. For example, if a user accepts an invitation to purchase lancets, the patient record 150 is checked to determine the frequency with which the patient has been instructed to check their blood glucose level. This allows the number of lancets needed for a given period to be calculated and presented.

The gathered and/or calculated order information, together with information from the solicitation such as pricing, is presented on the display of device 106 and the user is prompted to confirm the order (1024). If the order is not confirmed (1026), a prompt is presented asking if the user wishes to contact the DME provider (1028). If the user responds “no” (1034), a “no purchase” request is built (1038) and sent to the DME server 116 as previously described (1040), ending the method (1042). If the user requests contact with the DME provider (1034), a “DME contact requested” response is built (1036) and sent to the DME server 116 (1040), also ending the method (1042). For example, if the user cancelled the order because of questions regarding the order information presented, the user has the opportunity to request assistance from a DME provider representative. Once received by the DME provider, the request may be forwarded to a DME provider representative (e.g., via email) who can provide the user with the required assistance (not shown).

If the user confirms the order information (1026), the user is prompted to provide a password or other type of authentication data (1030), e.g., biometric data. Once the authentication data has been provided, the order information and authentication data are incorporated into a “purchase request” response (1032) which is sent to the DME server 116 (1040), ending the method (1042). In at least some examples of the system 100 the order information and confirmation are forwarded by the DME server 116 to a DME representative for further processing (not shown), while in other examples the purchase information and confirmation are both sent to an automated purchasing system (also not shown).

As noted earlier, FIG. 11 illustrates an example method 1100 performed by the DME server 116 for generating the invitations, advertisements and solicitations for the device 106, and for processing the responses provided by the device user. The example method 1100 of FIG. 11 may be performed periodically (e.g., daily) for a given patient record 150 stored on DME server 116, or upon detecting certain events associated with that patient (e.g., detecting a connection with that patient's device 106).

The record 150 is first read (1102) and checked to see if the user has authorized the DME provider to use information within patient record 150 (1104) to provide patient-specific, treatment-related promotions (see FIG. 2, “promotions allowed” field). If such use has not been authorized by the patient (1104), the patient record is skipped, ending the promotions processing for that patient (1146). If the patient has authorized the DME provider to access his/her patient data for promotional purposes (1104), the patient's record 150 is inspected to determine whether it is a new record or a previously existing record that has changed (1106). For example, the patient's profile will have changed if additional prescriptions from the patient's doctor are now on file, or existing prescriptions have been modified. To this end, patient record 150 may include a change flag (not shown) that is set whenever the record is modified.

If the patient record 150 has been newly created or modified (1106), a list of promotions associated with the patient record is built for a new record, or rebuilt for an existing record. These promotions may include the invitations, advertisements and/or solicitations previously described. The resulting patient's promotions list is stored in the patient's record 150 (see FIG. 2), and is tailored to the specific patient based at least in part on the patient's condition and/or treatment plan, as reflected in record 150. Thus, for example, if a blood glucose level meter is prescribed to the patient, the solicitation and advertisement selections will be based at least in part on the fact that the patient is diabetic, and may involve lancets, special socks, etc. as discussed with respect to FIG. 10.

After checking for a patient record creation/update (1106) and, if needed, building/rebuilding the patient's promotions list (1107), the DME server 116 determines whether it is time to send the patient a promotion for a specific product or service from the patient's promotions list (1108). This determination may be based on a flag within patient record 150 (not shown) indicating, for example, that method 1100 was invoked in response to a connection with the device. Alternatively, the determination may be based on the amount of time since a promotion was last sent to the patient's device 106 and the number of allowed promotions per time period specified by the patient (e.g., no more than one promotion per day) and stored in the patient record 150 (also not shown). If it is not time to send a promotion to the patient's device 106 (1108), that patient's record is skipped, ending the method (1146).

If it is time to send a promotion to the patient's device 106 (1108), a promotion is selected from the patient's promotions list. The selection of the promotion may be based on any of a number of mechanisms, including a random selection mechanism or a round-robin selection mechanism, just to name two examples. If an invitation to view the promotion is required (1112), the invitation is transmitted to the device 106 (1114) and presented to the user. Such invitations may be required, for example, by the user (see FIG. 2, “invitations required” field) and allow the user of the device 106 to forego viewing the promotion. If the user declines the invitation (1116), the method ends (1146). If the user accepts the invitation (1116), or if no invitation is required (1112), the promotion is transmitted to the device 106 (1122), e.g., as a video file or stream as previously described with regard to FIG. 10. At this point, the date at which the promotion was presented is logged into the patient's record 150 (see “promotion x (date)” in FIG. 2).

If the transmitted promotion is an advertisement (1124), no further processing is required and the method ends (1146). If the transmitted promotion is not an advertisement but instead a solicitation requiring further user input (1124), and after some delay (1126), a user response to the solicitation is received and logged in the patient's record 150 (see “user response x (date)” in FIG. 2) (1128). As previously noted, responses are tracked by DME server 116 for later use in refining the list of solicitations and advertisements presented to the user.

If a “no purchase” response was received, indicating that the user expressly declined the purchase request (1130), the method ends (1146). If a “purchase request” response was received (1132), the DME server 116 checks whether a prescription is needed for the requested product or service (1136), and if needed whether such a prescription is on file in the record 150 for the patient (1140). If no prescription is needed or a required prescription is on file, the purchase request is forwarded for further processing (1144), ending the method (1146). For example, the purchase request may be forwarded to a separate purchase processing system or to other purchase processing software executing on DME server 116. If a prescription is needed (1136) and the required prescription is not on file (1140), the requested product or service cannot be provided to the patient. A message requesting that the DME provider be supplied the required prescription is queued for transmission to the device 106 (1142), ending the method (1146).

If the purchase request was not confirmed (1132), the DME server 116 checks whether a “DME contact requested” response was received (1134). If the user has requested contact with a DME representative, the request is forwarded (e.g., to a customer service center) for further processing (1138), ending the method (1146). If the received message is not a request for DME contact from the user (1134), the message is not recognized and is ignored, ending the method (1146). Alternatively, the error may be logged in record 150 before ending the method (not shown).

Because the solicitations and advertisements presented are based at least in part upon information directly related to a patient's medical condition and/or prescribed treatment, the products and services advertised are more likely to be of use to the patient, and the DME provider is also likely to receive a higher percentage of orders. As previously noted, such targeting may be further refined by tracking which advertisements are viewed, as well as which products and services are ultimately purchased.

The use of the patient's information to identify treatment-related solicitations and advertisements require prior authorization by the patient, in compliance with applicable laws and regulations such as the Health Insurance Portability and Accountability Act of 1996 (HIPAA).

IV. Conclusion

Other variations and modifications to system 100 will become apparent to those of ordinary skill in the art once the above disclosure is fully appreciated. Thus, although blood glucose level meters are presented herein, other medical measurement devices (e.g., blood oxygen level meters) may benefit from system 100, as may a variety of non-medical devices perhaps subject to compliance verification by other governmental entities (e.g., aviation maintenance and test equipment subject to Federal Aviation Administration regulations).

Although the DME and Medicare servers 116 and 120 are shown as standalone servers, the functionality of each may be integrated into existing servers used for producing and processing the required regulatory data, for exchanging data and messages between a patient's device and a computer system operated by an authorized third party, and for processing purchase requests for DME provider products. Product and service promotions may be presented by providers other than the DME provider.

Also, although the examples shown thus far have been described within the context of a single-patient glucose meter, other examples may include multi-patient medical measurement devices, wherein at least some of the previously described collection, processing and delivery of required data to a governmental entity such as Medicare are performed on a per-patient basis. The information collected may be used to support claims submitted by a healthcare service provider to HHS, rather than (or in addition to) a claim by a DME. Such claims may be submitted by the healthcare service provider directly to HHS, or indirectly through a DME provider. Additionally, other information that may be required from a healthcare service provider, such as proof of proficiency of the operators of the device and/or calibration and quality control data of the medical measurement device, may also be provided to one or more governmental agencies. Examples of such agencies may include the United States Food and Drug Administration, and state medical professional licensing agencies. The data may also be used to obtain certifications required as a prerequisite to submitting claims for services provided using the medical measurement device, or to prove compliance with regulations governing the use of such devices by a service provider.

One skilled in the art will realize that the disclosed techniques are usefully implemented as software running on a computer system, and ultimately stored in a computer-readable medium, such as a magnetic or optical disk, a solid-state memory, etc. Such a computer system can be broadly construed as any machine or system capable or useful in reading and executing instructions of the software program. Ccomputer-readable media can comprise a single medium or multiple media (e.g., a centralized or distributed database, and/or associated caches and servers) that store the one or more sets of instructions.

It should also be noted that although the example methods described above and shown in the drawings indicate steps performed in a specific order, the claimed subject matter of the present disclosure is not limited to embodiments that perform such steps only in the order shown. Other alternative examples of these methods are contemplated that may perform steps in a different order.

What is claimed is:

1. A medical measurement device, comprising:
a data acquisition circuit for taking measurement data indicative of the user's health;
a communication interface for communicating with a medical measurement device provider's computer system, wherein the communication interface additionally provides the measurement data to the computer system; and
a user interface for allowing the user to receive and send messages from and to a third party via the communication interface.

2. The device of claim 1, wherein the device is handheld.

3. The device of claim 1, wherein the communication interface comprises a port on the device for coupling the device to a computer.

4. The device of claim 1, wherein the communication interface comprises a wireless communication interface.

5. The device of claim 1, wherein the measurement data comprises time-stamped glucose readings.

6. The device of claim 1, wherein the user interface comprises a display.

7. The device of claim 1, wherein the user interface comprises an alpha-numeric input device.

8. The device of claim 1, wherein the messages are text messages.

9. The device of claim 1, wherein the third party comprises the user's physician.

10. A medical measurement device, comprising:
a data acquisition circuit for taking measurement data indicative of the user's health;
a communication interface that receives one or more messages from, and transmits the measurement data to, a provider of medical measurement devices or of medical measurement device consumables; and
a display device that presents at least one of the one or more messages to a user operating the medical measurement device,
wherein the one or more messages comprise treatment-related information sent to the user from the third party.

11. The device of claim 10, further comprising an input device, wherein the user uses the input device to provide the measurement data in response to one of the messages.

12. The device of claim 10, wherein the treatment-related information comprises information selected from the group consisting of treatment changes, medical alerts, answers to user questions, and appointment information.

13. The device of claim 10, wherein the one or more messages comprises an authorized change to a configuration of the medical measurement device.

14. The device of claim 10, wherein the communication additionally transmits to the provider data indicative of the status and/or configuration of the medical measurement device.

15. A method implementable in a computer system for enabling communication between a user of a medical measurement device and a third party, comprising:
receiving at the computer system and from the medical measurement device measurement data indicative of the user's health;
receiving at the computer system an electronic message from a third party for the user;
verifying at the computer system whether the third party is authorized by the user by consulting a user record stored at the computer system;
if authorized, transmitting the received message to the medical measurement device.

16. The method of claim 15, wherein the measurement data comprises time-stamped glucose readings.

17. The method of claim 15, wherein the message comprises a text message.

18. The method of claim 15, wherein the third party comprises the user's physician.

19. The method of claim 15, wherein the message comprises information selected from the group consisting of treatment changes, medical alerts, answers to user questions, and appointment information.

20. The method of claim 15, wherein the message comprises an authorized change to a configuration of the medical measurement device.

21. A method implementable in a computer system for enabling communication between a user of a medical measurement device and a third party, comprising:
receiving at the computer system from a third party computer a first message for the user;
transmitting from the computer system the first message to the medical measurement device if the third party has been authorized by the user, wherein the medical measurement device is configured to take measurement data indicative of the user's health;
receiving at the computer system and from the medical measurement device a second message for the third party; and
transmitting from the computer system the second message to the third party computer.

22. The method of claim 21, wherein the measurement data comprises time-stamped glucose readings.

23. The method of claim 21, wherein the first and second messages comprises text messages.

24. The method of claim 21, wherein the third party comprises the user's physician.

25. The method of claim 21, wherein the second message comprises information selected from the group consisting of treatment changes, medical alerts, answers to user questions, and appointment information.
